(19) United States
(12) Patent Application Publication
(10) Pub. No.: US 2024/0423505 A1
(43) Pub. Date: Dec. 26, 2024

(54) RHEOLOGICALLY MODIFIED LIQUID METAL DEVICES AND RELATED SYSTEMS AND METHODS

(71) Applicant: THE REGENTS OF THE UNIVERSITY OF COLORADO, A BODY CORPORATE, Denver, CO (US)

(72) Inventor: Jianliang Xiao, Boulder, CO (US)

(21) Appl. No.: 18/750,876

(22) Filed: Jun. 21, 2024

Related U.S. Application Data
(60) Provisional application No. 63/522,934, filed on Jun. 23, 2023.

(51) Int. Cl. A61B 5/11; A61B 5/00

(52) U.S. Cl. CPC A61B 5/1126; A61B 5/6806; A61B 5/7264

(57) ABSTRACT

A liquid metal (LM) sensor is provided herein. In certain embodiments, the LM sensor includes a wire. In certain embodiments, the wire includes a LM composite material. In certain embodiments, the LM composite material includes a LM material and a nonconductive material.

### CROSS-REFERENCE TO RELATED APPLICATIONS

The present application claims priority under 35 U.S.C. § 119(e) to U.S. Provisional Patent Application No. 63/522,934, filed Jun. 23, 2023, the content of which is hereby incorporated by reference in its entirety herein.

### STATEMENT REGARDING FEDERALLY SPONSORED RESEARCH OR DEVELOPMENT

This invention was made with government support under grant number CMMI1762324 awarded by the National Science Foundation. The government has certain rights in the invention.

### BACKGROUND

Liquid metals (LMs) are highly desired in skin-mounted, deformable electronics due to their distinct combination of excellent electrical conductivity comparable to that of metals and exceptional deformability derived from its liquid state. Stretchable LM-based flexible electronics enable precise perception of complex strain and pressure stimuli, thus holds great promise in health monitoring, advanced wearable electronics, and human-machine interfaces. However, achieving sophisticated applications with LMs remains a daunting challenge, which requires overcoming the high surface tension of LMs and improving the sensitivity of sensors.

Liquid metals (LM) exhibit a distinct combination of high electrical conductivity (e.g., comparable to that of non-liquid metals) and exceptional deformability derived from its liquid state. Thus, LM may be considered a promising material for high-performance soft electronics. Stretchable LM-based flexible electronics enable precise perception of complex strain and pressure stimuli, thus holds great promise in health monitoring, advanced wearable electronics, and human-machine interfaces. However, achieving sophisticated applications with LMs can provide certain challenges. For example, overcoming the high surface tension of LMs and improving the sensitivity of sensors using LMs provides application challenges. Rapid patterning LM to achieve a sensory system with high sensitivity has posed a challenge, mainly attributed to the poor rheological property and wettability of LM materials. When metal particles are incorporated into LM's, both LM's and metal particles can be acid treated (e.g., to eliminate oxidation, to ensure homogeneous dispersion, etc.) which allows the particles to be wetted and suspended in the LM's (e.g., to improve adhesion for directly writing on various substrates). Ultrasonic treatment can be adopted to improve device manufacturing. In ultrasonic treatments, the accelerated oxides can be distributed inside LM's during sonication, to increase the viscosity of LM's. However, due to the limited viscosity improvement effect, it remains a challenge to pattern the LMs with rapid prototyping capabilities.

### SUMMARY

One aspect of the disclosure provides a liquid metal (LM) sensor. The LM sensor includes a wire. The wire includes a LM composite material. The LM composite material includes a LM material and a nonconductive material.

Another aspect of the disclosure provides a method of method of monitoring motion with sensor. The method includes (a) providing the wire to a portion of a human body to monitor motion; (b) applying an electrical signal through the wire; (c) monitoring the electrical signal; and (d) determining a motion profile based on material and geometric properties of the wire.

Another aspect of the disclosure provides a method of modifying rheological properties of liquid metal sensor. The method includes (a) providing a liquid metal (LM) material; and (b) integrating a nonconductive material into the LM material to form a LM composite material.

Another aspect of the disclosure provides a system for recognizing personal activities. The system includes a liquid metal (LM) sensor configured to measure motion or pressure. The LM sensor includes a wire made from a LM composite material. The LM composite material includes a LM material and a nonconductive material. The system also includes a computer system in electronic communication with the LM sensor. The computer system is configured to determine a plurality of motion profiles from motion signals from the LM device. The computer system includes a convolution neural network configured to be trained to differentiate each of the plurality of motion profiles.

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

The invention is best understood from the following detailed description when read in connection with the accompanying drawings. It is emphasized that, according to common practice, the various features of the drawing are not to scale. On the contrary, the dimensions of the various features are arbitrarily deformed or reduced for clarity. Included in the drawings are the following figures.

FIG. 1A illustrates schematics of the structure of embodiments of printable LM sensors integrated on boxing gloves, in accordance with exemplary embodiments of the present disclosure.

FIG. 1B illustrates schematics of exemplary LM sensors and multisensory feedback in connection with a deep learning algorithm, in accordance with exemplary embodiments of the present disclosure.

FIGS. 2A-2F illustrate rheology tests and 3D printing of LM-SiO₂ composites, in accordance with exemplary embodiments of the present disclosure.

FIGS. 3A-3F illustrate strain sensing of the LM sensor, in accordance with exemplary embodiments of the present disclosure.

FIGS. 4A-4H illustrate real-time monitoring of various human motions using the LM strain and pressure sensors, in accordance with exemplary embodiments of the present disclosure.

FIGS. 5A-5E illustrate a LM sensor for decoding the clenching posture and hitting strength of a punch in boxing training, in accordance with exemplary embodiments of the present disclosure.

FIGS. 6A-6F illustrate boxing identification of tactile glove assisted with deep learning, in accordance with exemplary embodiments of the present disclosure.

FIG. 7 illustrates a schematic of the fabrication process of LM-SiO₂ composites.

FIG. 8 illustrates a rheology test of liquid metal composites with 40 μm SiO₂ particles in amplitude sweep. (a)-(d) The amplitude sweep at 1 rad s⁻¹: (a) G′, G″ versus strain % where the G′=G″ is marked (empty circle); (b) G′ versus strain 0.05-1000% with the intersection of the regression lines used to calculate the limit of the linear viscoelastic region (LVR); (c) G′, G″ versus stress plot where G′=G″ is marked (empty circle); and (d) stress versus strain plot where the intersection of the regression lines mark the yield stress and strain (empty circle).

FIG. 9 illustrates a rheology test of liquid metal composites with 40 μm SiO₂ particles in frequency and flow sweeps. (a) The frequency sweep plot of G′, G″ over frequency at 0.5% strain. (b) The flow sweep data plotted as viscosity over shear rate. (c) Stress growth at 0.01 s⁻¹ producing plot of stress versus strain where the regression line intercept point marks the yield stress and strain (empty circles).

FIG. 10 illustrates a rheology test of liquid metal composites with 6 μm SiO₂ particles in amplitude sweep. (a)-(d) The amplitude sweep at 1 rad s⁻¹: (a) G′, G″ versus strain 0.05-1000% where the G′=G″ is marked (empty circle); (b) G′ versus strain 0.05-1000% with the intersection of the regression lines used to calculate the limit of the linear viscoelastic region (LVR); (c) G′, G″ versus stress plot where G′=G″ is marked (empty circle); and (d) stress versus strain plot where the intersection of the regression lines mark the yield stress and strain (empty circle).

FIG. 11 illustrates a rheology test of liquid metal composites with 6 km SiO₂ particles in frequency and flow sweeps. (a) The frequency sweep plot of G′, G″ over frequency at 0.5% strain. (b) The flow sweep data plotted as viscosity over shear rate. (c) Stress growth at 0.01 s⁻¹ producing plot of stress versus strain where the regression line intercept point marks the yield stress and strain (empty circles).

FIG. 12 illustrates SEM images of liquid metal composites with 40 km SiO₂ particles, with different filler loadings. (a) 0 wt. %, (b) 1 wt. %, (c) 3.5 wt. %, (d) 6 wt. %.

FIG. 13 illustrates SEM images of liquid metal composites with 6 km SiO₂ particles, with different filler loadings. (a) 1 wt. %, (b) 3.5 wt. %, (c) 6 wt. %, (d) 20 wt. %.

FIG. 14 illustrates the schematic fabrication process of LM-based sensor. (a) Plain view, (b) 3D view.

FIG. 15 illustrates a comparison of the resistance change between theoretical calculation and experimental test.

FIG. 16 illustrates the distribution of SiO₂ particles in LM wires.

FIG. 17 illustrates experimental data and fitting curve of stress-strain relationship of Ecoflex.

FIG. 18 illustrates a liquid metal processing and printing setup. (a) 3D printer setup; (b) 3D printer extruder head with an attached 10 mL syringe.

FIG. 19 illustrates a table of liquid metal processing and printing settings.

### DETAILED DESCRIPTION OF THE INVENTION

The present disclosure provides a liquid metal (LM) sensor. The LM sensor comprises a composite including a LM material and a nonconductive material. In some embodiments, the nonconductive material is distributed throughout at least a portion of the LM material. For example, the nonconductive material may include nonconductive particles that are mixed with and/or distributed throughout the LM material. In some embodiments, the nonconductive particles are uniformly distributed throughout the LM material.

The LM material includes any suitable low melting temperature metal or metal alloy. As used herein, the term low melting temperature refers to any metal or metal alloy with a melting temperature below 80° C. Suitable LM materials include, but are not limited to: a gallium alloy; an indium alloy; a gallium-indium alloy; Eutectic gallium-indium (EGaIn); a Galinstan (GaInSn) alloy; a Field's alloy; and a Bismuth based Eutectic alloy.

The nonconductive material includes any suitable material or combination of materials that is nonconductive or substantially nonconductive. Suitable nonconductive materials include, but are not limited to: oxide particles; polymer coated conductive particles; polymer coated nonconductive particles; oxide coated conductive particles; oxide coated nonconductive particles; and polymer particles. For example, in some embodiments, the nonconductive material includes SiO₂ particles. In other embodiments, the nonconductive material includes TiO₂ or Al₂O₃ particles (or similar oxides). In certain embodiments, the nonconductive material can include a plurality of particles (e.g., Si SiO₂ and TiO₂ particles).

In certain embodiments, the nonconductive material includes the a plurality of nonconductive particles with a particle size within the range of 10 nm-500 μm. In certain embodiments, the particle size (e.g., of SiO₂) can be 6 μm or 40 μm.

In some embodiments, the incorporation of the nonconductive material in the composite modifies the rheological properties and/or strain redistribution mechanics of the composite. For example, a plurality of particles (of the nonconductive material) can increase the effective viscosity of the LM composite material (as compared to a base or unmodified LM material).

In some embodiments, the LM sensors can be positioned on different locations of a human body for wearable applications (e.g., personal fitness applications). In such embodiments, when stretched, the strain redistribution mechanics provided by the LM composite disclosed herein can lead to greatly enhanced gauge factor. Sensors based on printed LM composite material (e.g., LM-SiO₂ composite) provide excellent mechanical flexibility, robustness, strain, and pressure sensing performances.

In certain embodiments, the LM sensors described herein can be integrated into one or more articles for wearable applications on different locations of a human body. By integrating such sensors into the one or more articles, such as a tactile glove, the synergistic effect of strain and pressure sensing can decode the clenching posture and hitting strength in boxing training. When assisted by a deep learning algorithm, such a tactile glove can achieve recognition of the technical execution of boxing punches, such as jab, swing, uppercut, and combination punches (e.g., with 90.5% accuracy). The integrated multifunctional sensory system described herein can find wide applications in smart sport-training, intelligent soft robotics, and human-machine interfaces.

LM sensors of the present disclosure improve sensitivities (e.g., as compared to existing LM sensors). For example, the strain redistribution mechanics provided by the nonconductive material (e.g., SiO₂ particles, SiO₂ micro-particles, etc.) allow an LM sensor (e.g., a printed sensor) to achieve high sensitivity (e.g., gauge factor is 5.72 for strain range within 100%; 11.36 for 100-200%; 23.91 for 200-300%; etc.) and excellent robustness. LM-based strain sensors can be based on piezoresistive effects, due to a change in electrical resistance upon deformation. The gauge factor of a piezoresistive strain sensor attributes can be a function of: (1) a geometry change due to deformation; and (2) a resistivity change. The resistivity of LM's can remained unchanged due to their excellent conductivity properties; thus resistance change of LM's only (or at least primarily) depends on their geometry changes during deformation. Reported gauge factors of LM-based strain sensors are usually in the range of 2-6, thereby limiting their application when high sensitivity is desired (or required), such as in skin prosthetics, humanoid robotics, wearable health monitoring, and the like. It is highly desirable to develop novel sensory systems based on LM's with high sensitivity.

In one example, the LM sensors are integrated with a tactile glove, providing synergistic strain and pressure sensing capable of decoding certain information used in boxing training (e.g., the clenching posture, hitting strength, motion profiles, gesticulations, etc.). In certain embodiments, the conductive traces based on LM and nonconductive materials can withstand very high pressure, which can provide improved robustness of a LM device (e.g., a LM sensor, an apparatus including a LM sensor, a glove including an LM sensor, etc.). The embodiments of the present disclosure provide advantages in many applications and industries, such as wearable systems, sports training, soft robotics, and many others.

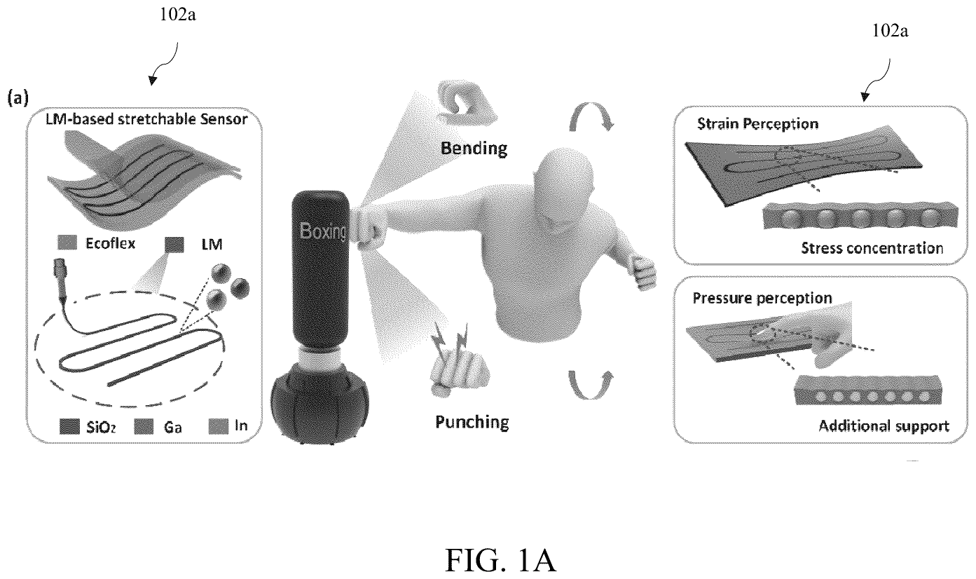

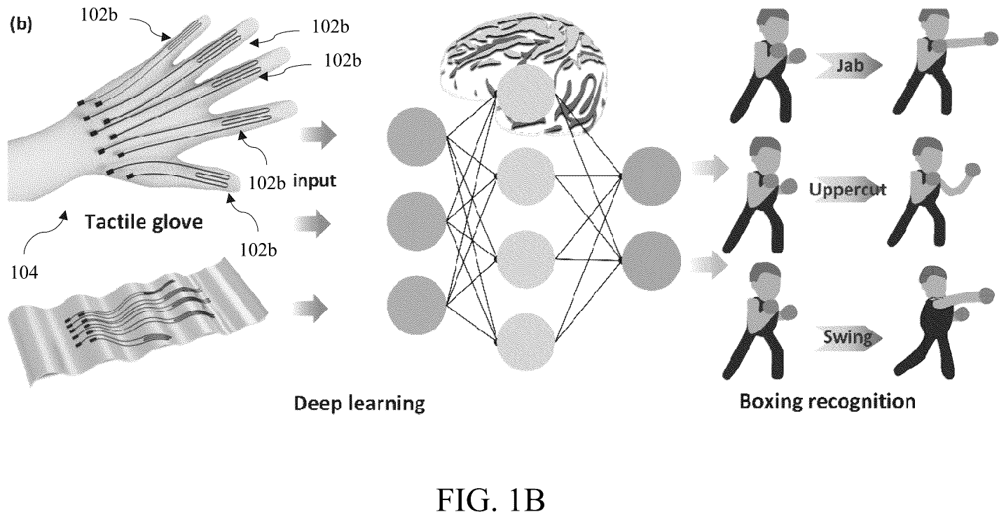

In certain embodiments, sensory arrays (including one or more LM sensors) can be further integrated into a tactile glove to exploit the capability of monitoring clenching postures and punching strength in real time. Combined with a trained convolutional neural network algorithm, a multifunctional tactile glove can classify the various boxing motions (e.g., a jab, swing, uppercut, combination punches, etc.) with high accuracy (e.g., up to and including 90.5%). FIG. 1B illustrates some of such boxing motions. Such aspects described herein demonstrate the capability of recognizing personal or physical activities (e.g., in boxing training). In accordance with exemplary embodiments of the present disclosure, a discussion of certain results of experiments and/or embodiments is provided herein.

Certain embodiments are best described in connection with the drawings. Referring now to the drawings, FIGS. 1A-1B illustrates printable liquid metal sensors 102a/102b and a tactile glove 104 with boxing recognition capability. FIG. 1A illustrates a schematic illustration of the structure of LM sensor 102a (e.g., a printable LM sensor). FIG. 1B illustrates a schematic a plurality of LM sensors 102b integrated with a glove 104 (e.g., a tactile glove, a boxing glove, etc.). The strain redistribution mechanics and enhanced pressure resistance due to the added nonconductive particles (e.g., SiO₂ particles) in the LM render the flexible sensors high sensitivity and robustness, thus perceiving strain and pressure stimuli during bending and punching process in the boxing sport. FIG. 1B illustrates the multisensory feedback enables the tactile glove to achieve boxing recognition, assisted with a deep learning algorithm.

The present disclosure also provides a rheological modification strategy of LM and strain redistribution mechanics to simultaneously simplify the scalable manufacturing process and to significantly enhance the sensitivity and robustness of LM sensors (e.g., LM-based strain and/or pressure sensors; see FIG. 1A). In some embodiments, incorporating the nonconductive material (e.g., silicon dioxide (SiO₂) particles, SiO₂ micro-particles, etc.) into the LM material drastically enhances and/or improves the effective material properties (and/or fluidic properties), as compared to the LM material alone. For example, improved viscosity (e.g., dynamic viscosity, kinematic viscosity, etc.), modulus (e.g., bulk modulus of elasticity, shear modulus, etc.), and yield stress (e.g., oscillation yield stress, shear yield stress, etc.). Additionally or alternatively, in some embodiments, the modified rheology of mixed LM with nonconductive particles improves the “printability” (i.e., the feasibility of use in certain “3D printing” or additive manufacturing processes) of such materials and simplifies associated fabrication processes.

The following examples further illustrate aspects of the present invention. However, they are in no way a limitation of the teachings or disclosure of the present invention as set forth herein. For example, although certain results are described in connection with an LM sensor using a LM composite material of LM-SiO₂ (e.g., using a GaIn alloy), the teachings of the results are applicable other LM composite materials (including other LM's and/or other nonconductive materials) described herein.

### EXAMPLES

### Rheological Modification of the Liquid Metal

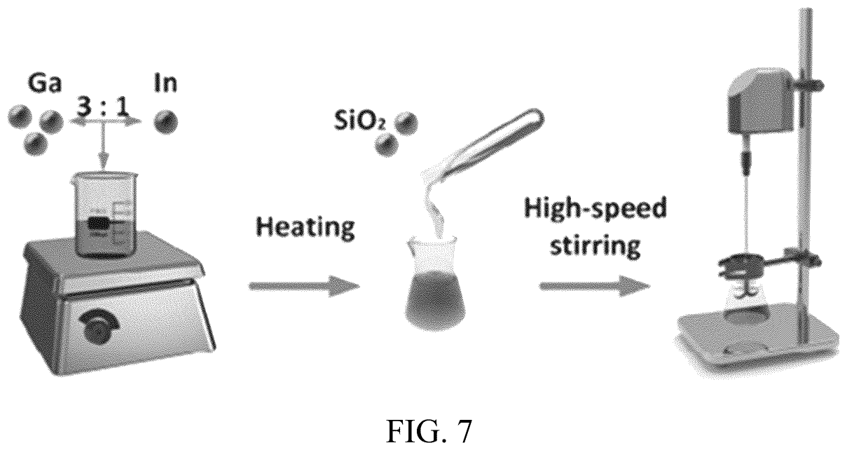

In certain embodiments, gallium-indium alloys (Ga:In 75:25 wt %) can be used as a LM material (e.g., conductive LM, a base LM material, etc.) and a nonconductive material (e.g., an additive material, nonconductive particles, nonconductive SiO₂ particles, etc.) can be mixed into the LM material to modify the material properties (e.g., mechanical properties, electrical properties, fluidic properties, etc.). An exemplary schematic of a fabrication process of an LM composite material (including the conductive LM material and the nonconductive material) is illustrated in FIG. 7.

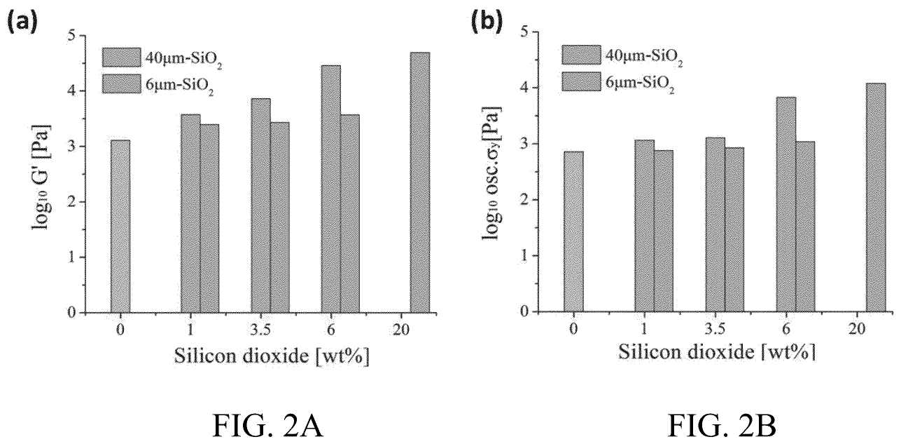

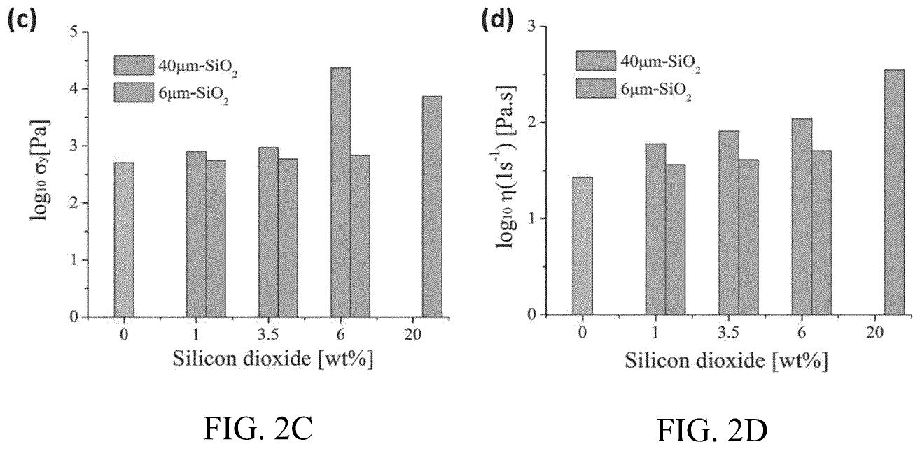

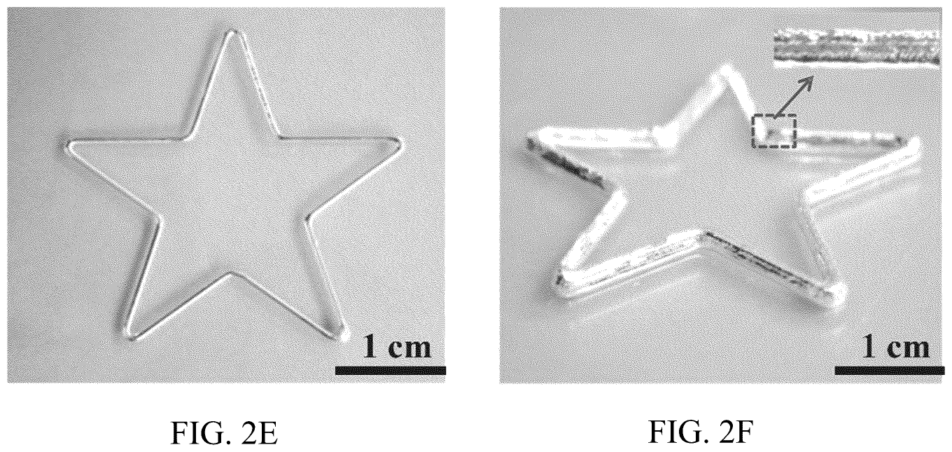

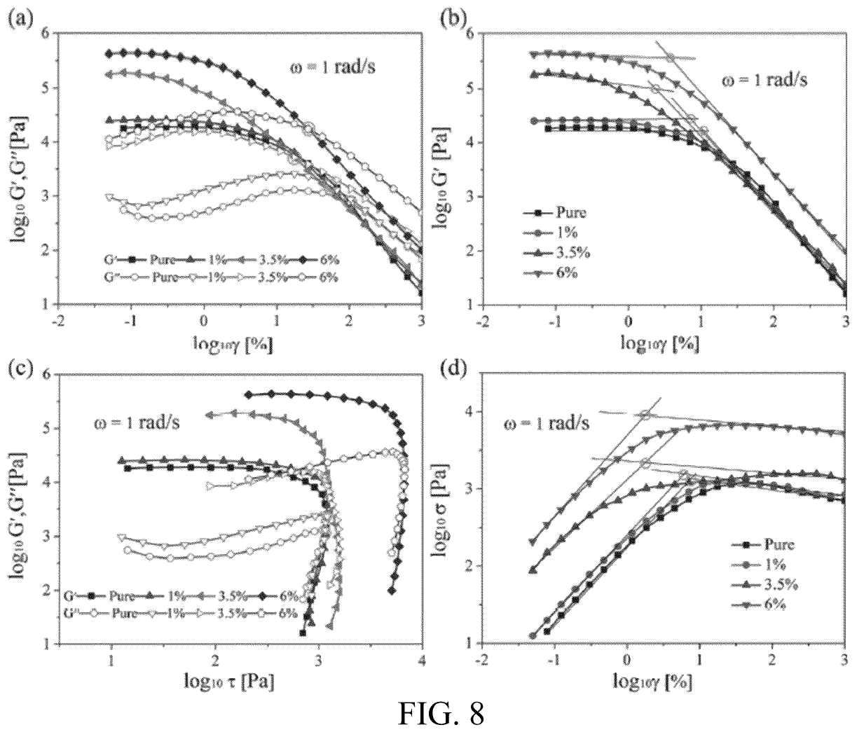

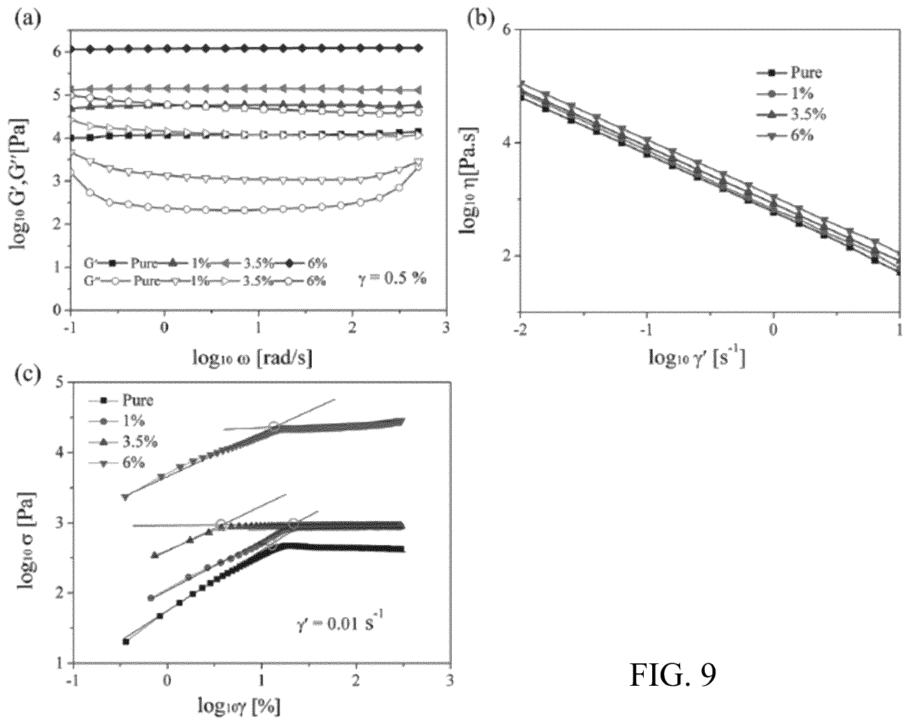

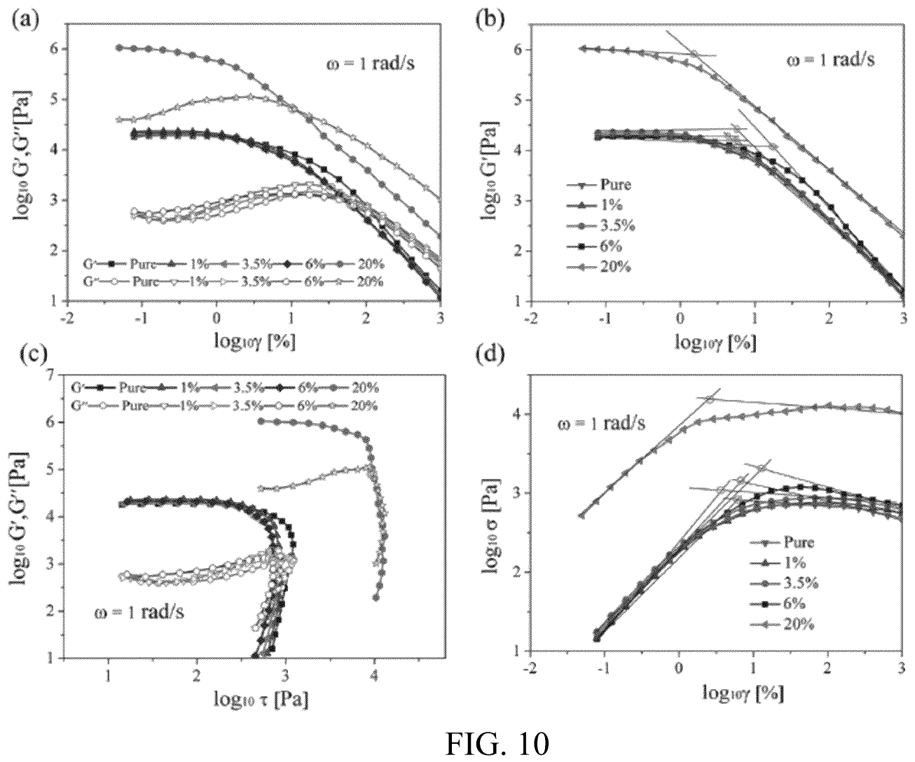

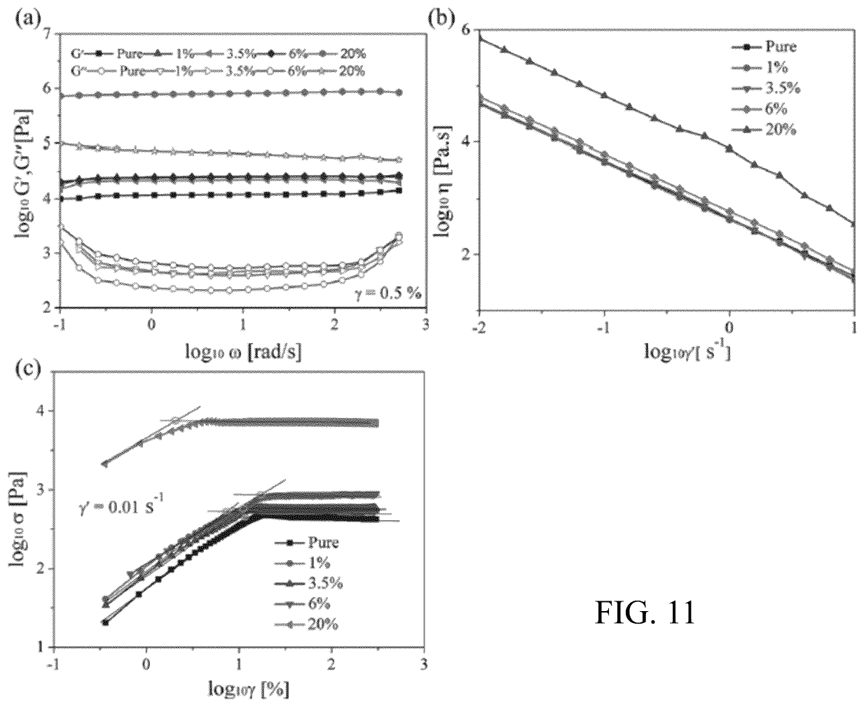

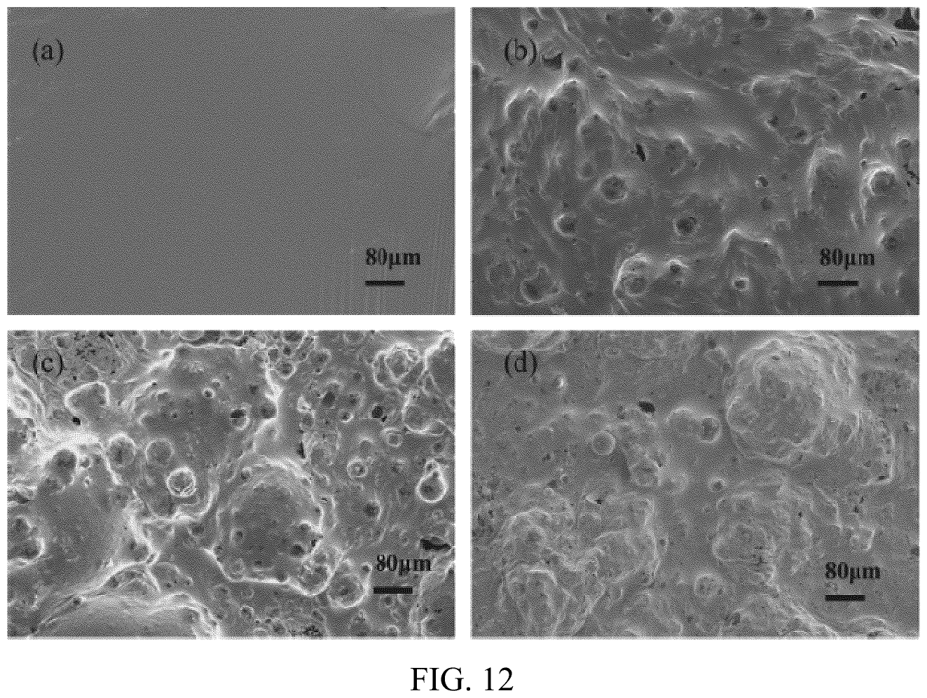

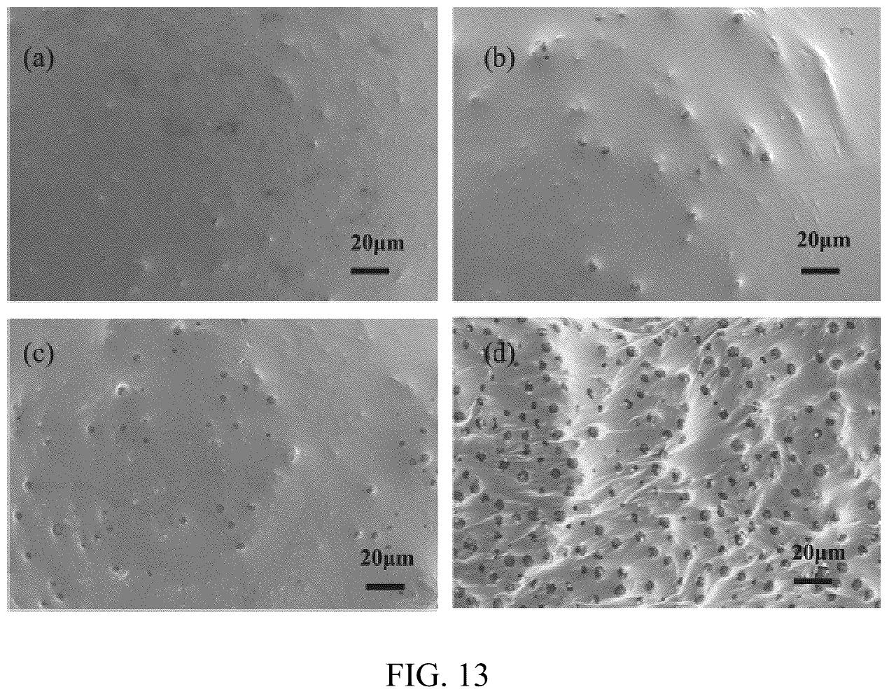

Different sizes of nonconductive material particles (e.g., SiO₂ particles) can be used. For example, in certain experiments and/or embodiments described herein, two different sizes of SiO₂ particles, 40 μm and 6 μm, are used for the LM composites. The rheological test results of certain experiments of the present disclosure are illustrates in FIGS. 8-10. FIGS. 8-10 illustrate that as the weight fraction of SiO₂ increases, the average elasticity, oscillation yield stress, shear yield stress, and the viscosity at 1 s⁻¹ all increase for both LM composites (e.g., see FIGS. 2A-2D). At the same weight ratios, the effect of rheological property modification caused by 40 μm particles is better than 6 μm particles. This may be due to the highly viscous medium (e.g., liquid gallium indium alloy) creating a kinetic barrier to interactions among particles (see FIGS. 12-13), and larger particles overcoming the barrier more easily when ink is flowing. The increased elastic modulus, shear stress, and viscosity enable the composites to remain in shape after extrusion, rendering it “3D printable.”

FIGS. 2A-2F illustrate rheology tests and 3D printed structures of LM-SiO₂ composites. FIG. 2A illustrates the average elastic modulus over different wt % of SiO₂ particles and the sizes of the particles (i.e., 40 μm particles and 6 μm particles). FIG. 2B illustrates yield stresses derived from the oscillation amplitude sweep over different wt % of SiO₂ particles and the sizes of the particles (i.e., 40 μm particles and 6 μm particles). FIG. 2C illustrates the yield stress derived from the stress growth test over different wt % of SiO₂ particles and the sizes of the particles (i.e., 40 μm particles and 6 am particles). FIG. 2D illustrates viscosity at a shear rate of 10 s⁻¹ derived from the flow sweep test over different wt % of SiO₂ particles and the sizes of the particles (i.e., 40 am particles and 6 am particles). The shear strength/stress can be described as the onset of the liquid starts to flow. For water (e.g., pure ware), shear strength/stress is zero. A viscous liquid needs some stress to break the resistance in order to flow. FIGS. 2E-2F illustrate photographs of 3D printed liquid metal structures of a one-layer star and a four-layer star, respectively.

The rheologically modified composites with 1 wt. % 6 μm SiO₂ particles were used to print complicated 3D structures (e.g., a one-layer star as illustrated in FIG. 2E and a four-layer star as illustrated in FIG. 2F), via dispensing the composites layer by layer. The printed conformations remained stable due to the structural support provided by the composites. Such stability opens up new opportunities to manufacture better multifunctional stretchable electronics in an efficient way.

### Characterization of the LM Strain and Pressure Sensors

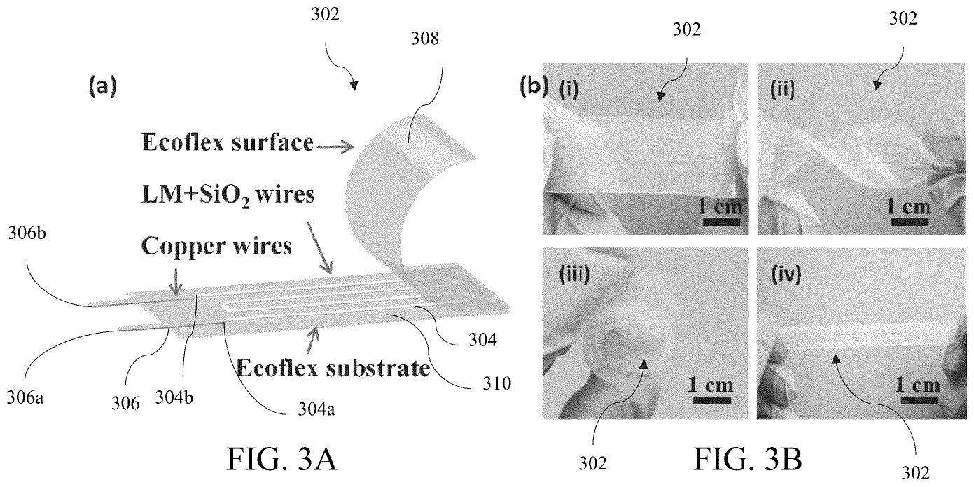

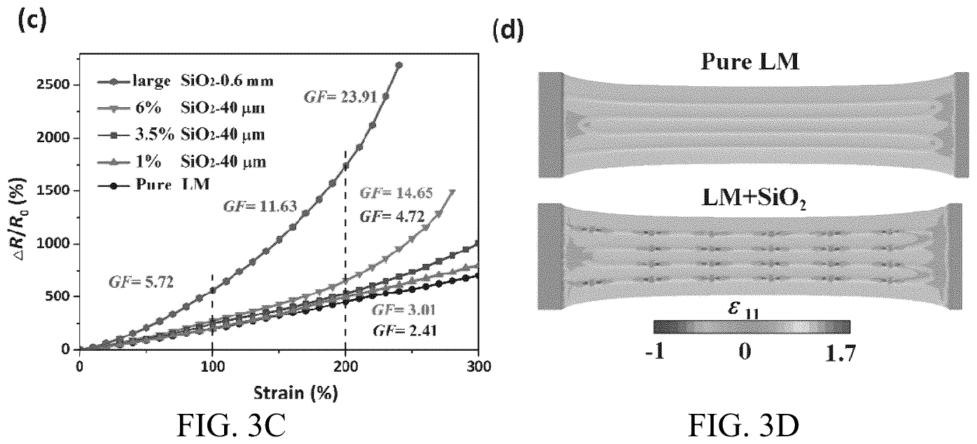

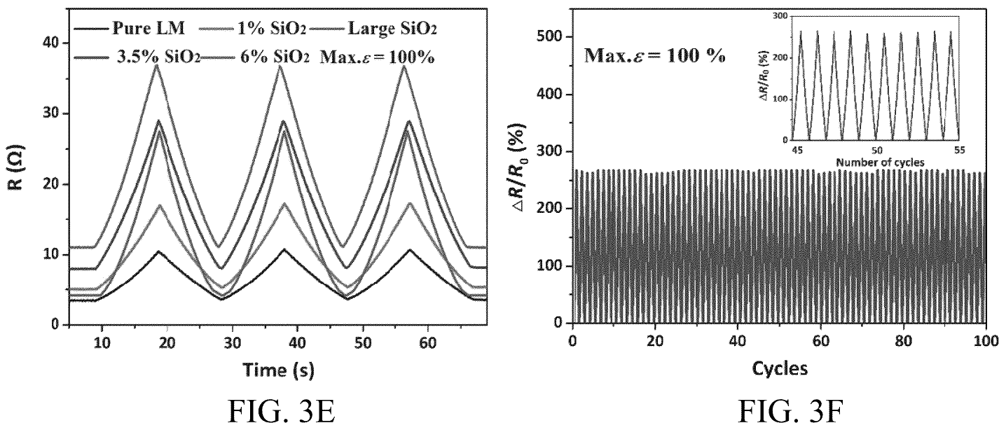

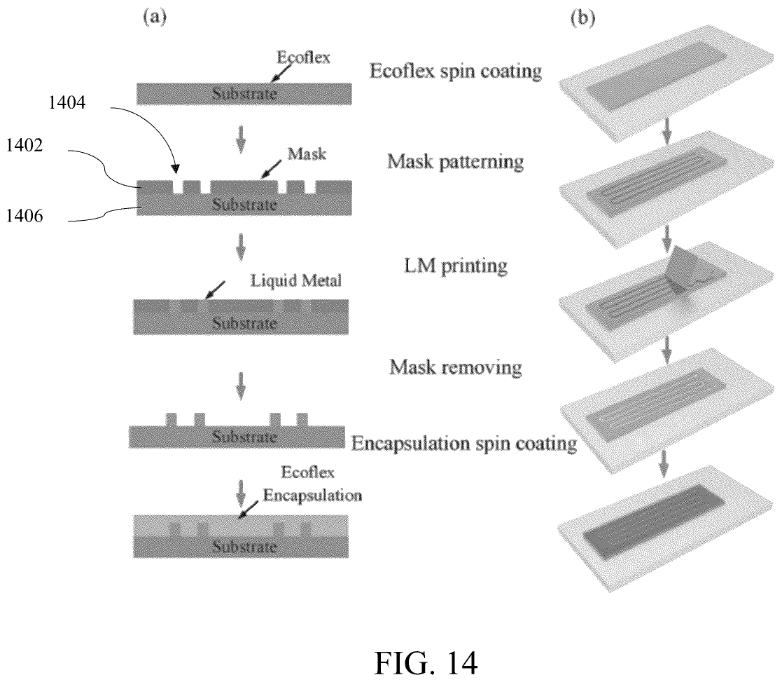

To further quantitatively evaluate the electrical and mechanical properties of the LM composite material (e.g., LM-SiO₂ microparticle composites), patterned composite wires were connected to copper tapes, and then encapsulated by Ecoflex to assemble sensing devices (see FIG. 3A and FIG. 14).

FIGS. 3A-3F illustrate strain sensing of an LM sensor 302. FIG. 3A illustrates a schematic illustration of the structure of LM sensor 302 (e.g., strain sensor). LM sensor 302 includes a wire 304 made from a LM composite material. Wire 304 is illustrated in a serpentine (e.g., an “S-shape” and/or an “M-shape”) geometry. LM sensor 302 includes one or more wires 306 (e.g., copper wire), including a first terminal 306a and a second terminal 306b. A first terminal 304a and a second terminal 304b of wire 304 are illustrated connected (e.g., electrically connected, conductively connected, fused, soldered, formed, etc.) to first terminal 306a and second terminal 306b, respectively. It should be understood that an electrical signal (e.g., current, voltage, impedance, etc.) can be provided (e.g., using one or more terminals) such that an electrical characteristic (e.g., current, voltage, impedance, etc.) of LM sensor 302 can be monitored. Such an electrical characteristic can be used to determine a certain material properties of LM sensor 302 (e.g., a dynamic material property of wire 304). LM sensor 302 is thus configured to sense strain (e.g., due to the strain-gauge type arrangement of wire 304 and wire(s) 306).

LM sensor 302 includes one or more of a substrate 308 and a substrate 310 (e.g., a flexible substrate, an “ecoflex” substrate, etc.). Substrate 308/310 can be used as a containment element to contain (or enclose) wire 304 and at least a portion of wire 306. Substrate 308/310 can be used as an attachment mechanism (e.g., configured to attach the wire to a human to monitor movement or pressure). In FIG. 3A, substrate 308 is an “upper” or “top” substrate and substrate 310 is a “lower” or “bottom” substrate. It should be understood that wire 304 can be formed (e.g., 3D printed) on a portion of a substrate (e.g., substrate 310). For example, wire 304 can be formed (e.g., 3D printed) in a channel defined (at least in part) by a substrate (e.g., see channel 1404, defined in part by a substrate 1406 and a mask 1402 in FIG. 14). Thus, a channel can substantially define the width or cross-sectional geometry of wire 304. A channel (e.g., of substrate 308/310) can act as a containment mechanism to contain wire 304. For example, in certain embodiments, wire 304 can be in a liquid or semi-liquid state, where one or more of substrate 308/310 and/or a channel function as a containment mechanism to hold wire 304 in place. In certain embodiments, wire 304 can be in a solid or semi-solid state, where one or more of substrate 308/310 and/or a channel function as a containment mechanism to hold wire 304 in place, but are not necessary to give wire 304 its shape.

FIG. 3B illustrates optical images of LM sensor 302 under different deformation modes: (i) initial state, (ii) twisted, (iii) rolled, and (iv) stretched. FIG. 3C illustrates relative change of electrical resistance versus strain of the LM sensors with different SiO₂ filler loadings. FIG. 3D illustrates mechanical FEA simulation results of strain distributions within the LM sensors based on pure LM (top image) and LM-SiO₂ composite (bottom image) under 100% tensile strain. FIG. 3E illustrates reversible loading-unloading behavior of the LM sensors with different SiO₂ filler loadings. FIG. 3F illustrates relative resistance change during cyclic stretching-releasing test.

The LM sensor 302 (e.g., strain sensor) can be twisted, curled, and stretched (as illustrated in FIG. 3B) without causing any damage to LM sensor 302. The experiments and embodiments described herein demonstrate excellent mechanical flexibility and stretchability.

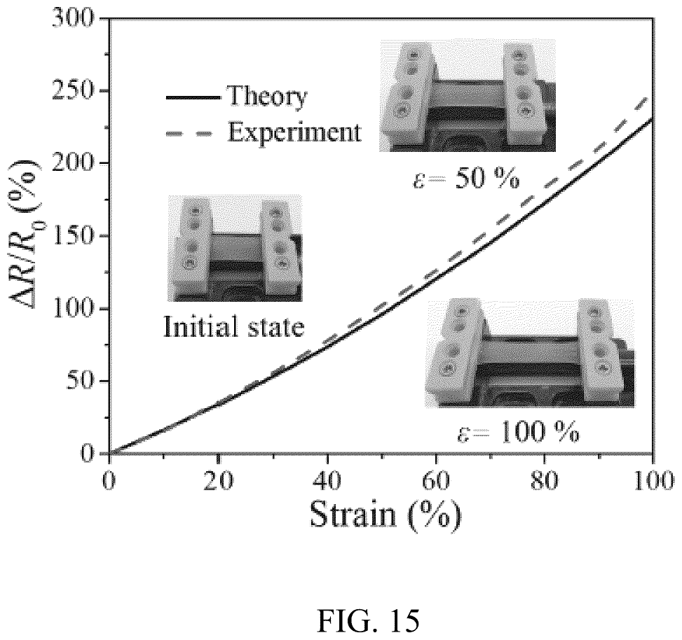

The gauge factor of a piezoresistive strain sensor is defined as, GF=ΔR/(R₀ε)=(1+2ν)+Δρ/(ρε), where ΔR is the resistance change, R₀ is the original resistance, ν is the Poisson ratio, ε is the applied tensile strain, Δρ is the resistivity change, and ρ is the original resistivity. The theoretical relationship between the resistance change and strain indicates that increasing the strain applied to pure LM wires leads to increase in electrical resistance, which is further confirmed by the experimental measurement under uniaxial tensile strain (see FIG. 15). FIG. 3C illustrates the relative resistance change of the strain sensor versus the applied tensile strain for strain sensors based on pure LM and LM-SiO₂ composites. For strain sensors based on pure LM, the relative resistance change shows linear increase with strain, with constant GF of 2.41. When 1 wt. % and 3.5 wt. % of 40 μm SiO₂ particles are mixed with the LM, the GF increases to 3.01 and 4.72, respectively. When the weight ratio of m SiO₂ particles increases to 6%, the curve exhibits highly nonlinear phenomenon at high strain level, leading to an effective GF of 14.65 over the strain range between 200% and 300%, which is more than six times higher than pure LM strain sensor.

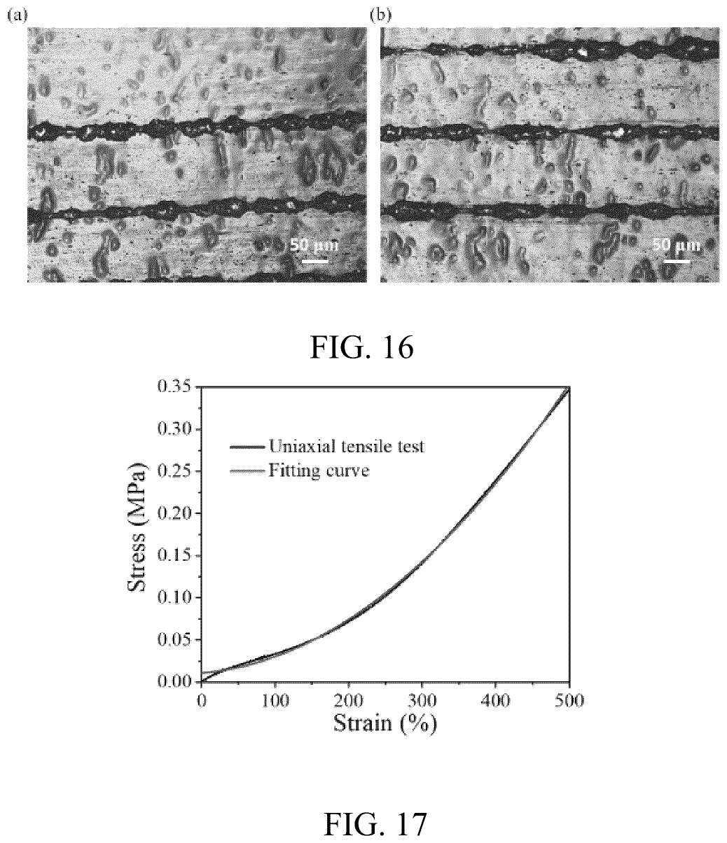

To elucidate the underlying mechanism of enhanced sensitivity due to incorporation of SiO₂ particles, finite element analysis (FEA) is used to compare the mechanical deformation of the LM sensors. It is assumed that SiO₂ particles are uniformly distributed in liquid metal wires based on experimental observation (see FIG. 16). FIG. 3D illustrates that the strain in the sensor based on pure LM is uniform throughout the sensor when deformed. But for the LM sensor based on LM-SiO₂ composite, strain redistribution occurs along the LM wires, causing localized narrowing of LM channels. Such LM channel narrowing induced rapid increase in electrical resistance and thus the enhancement in sensitivity. Inspired by the simulation results, we further explore the sensitivity enhancement of the strain sensor by increasing the SiO₂ particle size to 0.6 mm, which is close to the width of the LM wires (0.9 mm). At large strains, significant stress concentration could be induced around the particles, and because of the blockage of the conductive path around the particles, rapid increase in effective resistance and thus gauge factor could be achieved. As illustrated in FIG. 3C, the GF of LM sensor 302 (e.g., a LM strain sensor) using 0.6 mm SiO₂ particles increases from 5.72 between 0 and 100% strain to 11.63 between 100% and 200% strain, and further to 23.91 between 200% and 300% strain, which is almost 10 times enhancement from pure LM. Besides the high sensitivity, stability and durability are of great importance for practical applications of strain sensors.

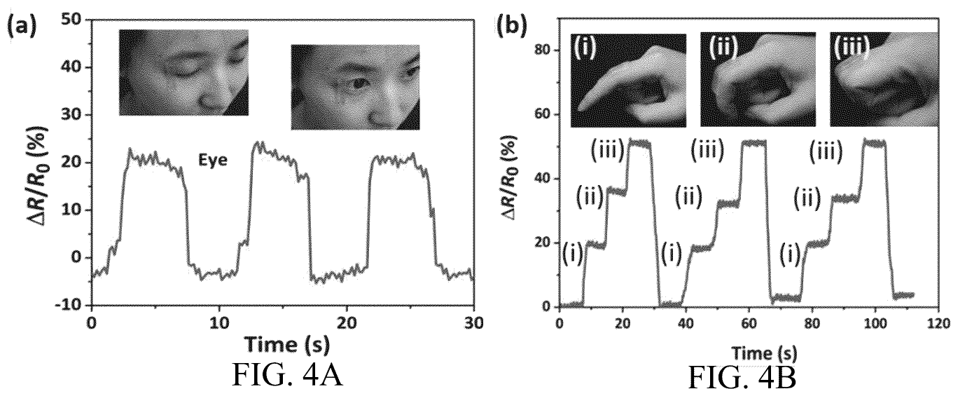

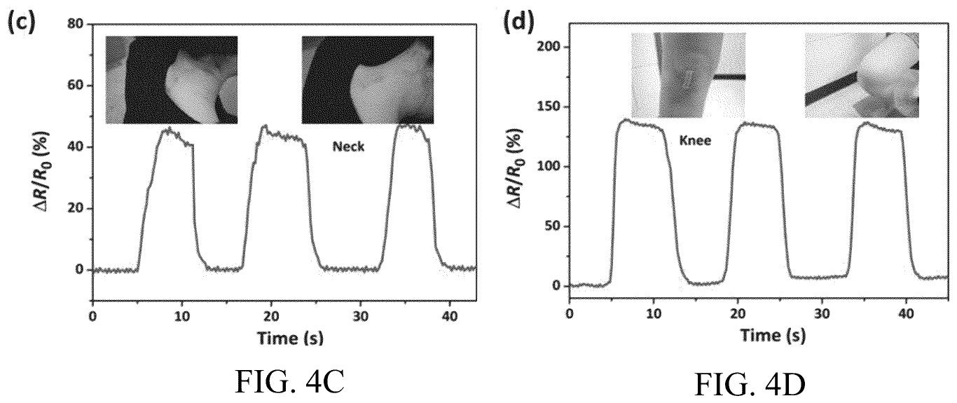

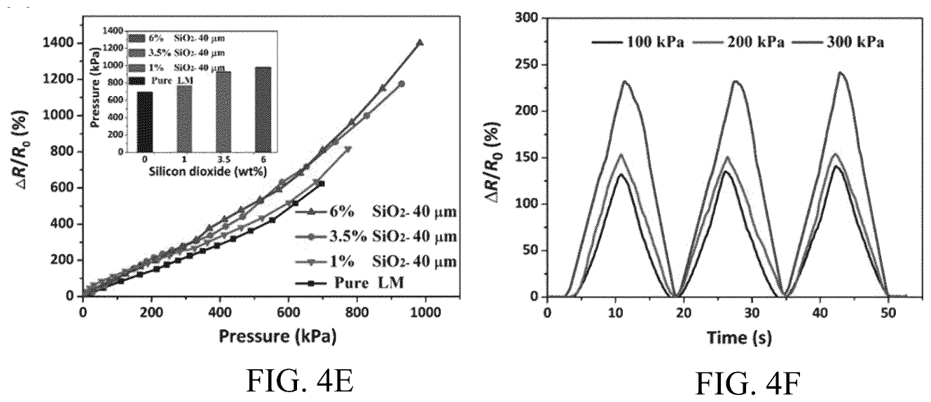

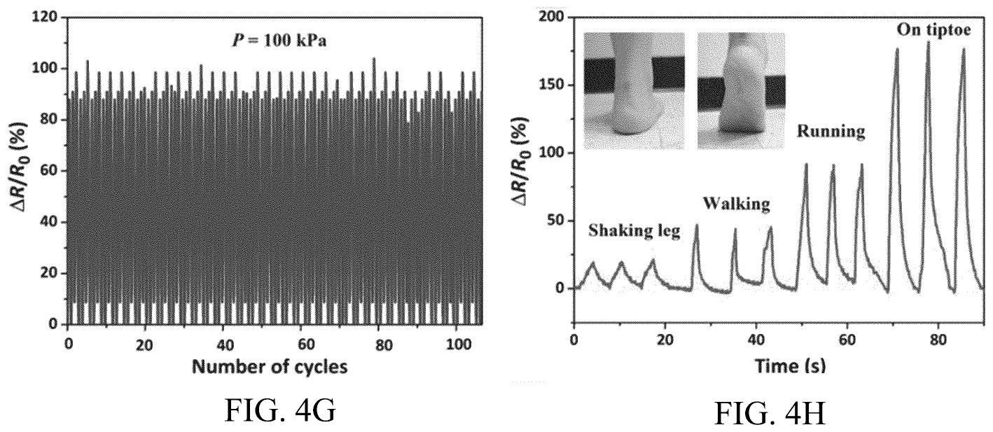

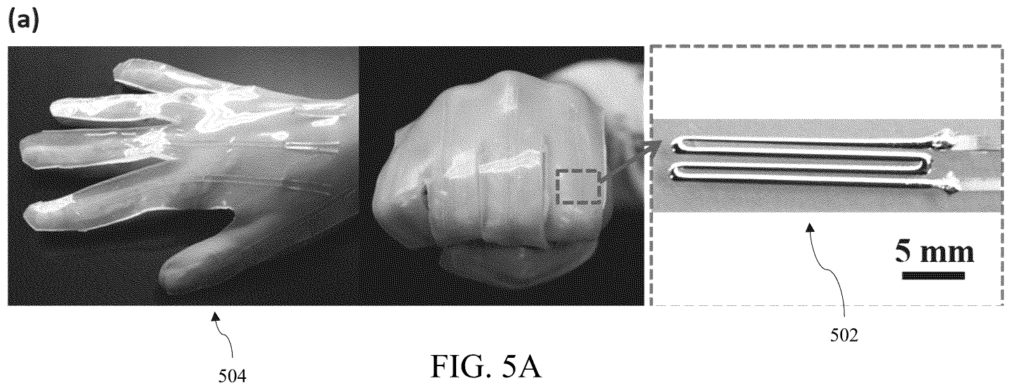

Cyclic loading and unloading testing of our strain sensors at 100% strain were conducted, and the results are illustrated in FIGS. 3E and 3F. As illustrated in FIG. 3E, strain sensors with different filler loadings show excellent reversibility and repeatability, with negligible hysteresis, due to the great elasticity of Ecoflex and flowability of LM-SiO₂ composites. Moreover, the electrical response of the strain sensors exhibits excellent stability during 100 stretching-releasing cyclic test under 100% strain (see FIG. 3F). Due to the excellent mechanical flexibility, high sensitivity at both small and large strains, and the extremely wide sensing range, the LM strain sensors can be used as a wearable device on human to monitor subtle and large motions at different locations in real time. The LM strain sensors are attached to the eye, neck, fingers, and knees of the human body to detect various human motions (see FIGS. 4A-4D). Not only the subtle muscle movements induced by micro-expression can be decoded from the analysis of the measured piezoresistive signals, but also the vigorous bending and releasing of the knee joint could be precisely recorded. Such demonstrated capabilities enable the LM sensor to be integrated with various objects to create application opportunities in intelligent soft robotic systems, interactive wearable electronics, and future human-machine interfaces.

In addition to strain sensing, LM sensors described herein can be used in pressure detection. FIG. 4 illustrates real-time monitoring of various human motions using the LM strain and pressure sensors. FIGS. A-D illustrate applications of the strain sensor as a human joint motion decoder in real time on/near: an eye, finger, neck, and knee, respectively. FIG. 4E illustrates relative changes of electrical resistance versus pressure of the LM pressure sensors with different SiO₂ filler loadings. FIG. 4F illustrates a reversible loading-unloading behavior of an exemplary LM sensor under 100 kPa, 200 kPa, and 300 kPa pressures. FIG. 4G illustrates relative resistance changes under cyclic compressing-releasing tests. FIG. 4H illustrates an application of an LM sensor (e.g., a pressure sensor) as a human plantar pressure decoder.

FIG. 4E illustrates the relative resistance change versus the pressure applied to the LM sensor. When the pressure exceeds a certain threshold, the LM sensor fails due to the collapse of conductive LM wires. The results indicate that the maximum pressure the sensor can withstand enhances from 696.3 kPa to 981.5 kPa as the SiO₂ weight fraction increases from 0% to 6% (inset of FIG. 4E). The incorporation of SiO₂ particles can provide more resistance to counter the compressive force, resulting in improvement in the detectable pressure range. FIG. 4F illustrates a reversible loading-unloading behavior of a LM sensor with 6% SiO₂ particles for the applied pressure ranging from 100 kPa to 300 kPa. The relative resistance changes of the sensor under cyclic compressing-releasing tests of 100 kPa pressure for 100 cycles (as illustrated in FIG. 4G) demonstrate excellent durability and repeatability. To illustrate the application in wearable electronics, the LM pressure sensor is attached to the bottom of a foot. FIG. 4H illustrates shows that the LM sensor can detect and distinguish different motions of the foot, such as shaking leg, walking, running, and tiptoeing, and therefore could be used for gait identification.

### Deep-Learning-Assisted Boxing Recognition

Precisely capturing the real-time strain and pressure information creates application opportunities for an LM sensor in boxing training, which can be utilized to provide sensory feedback for athletes to optimize the punching technique.

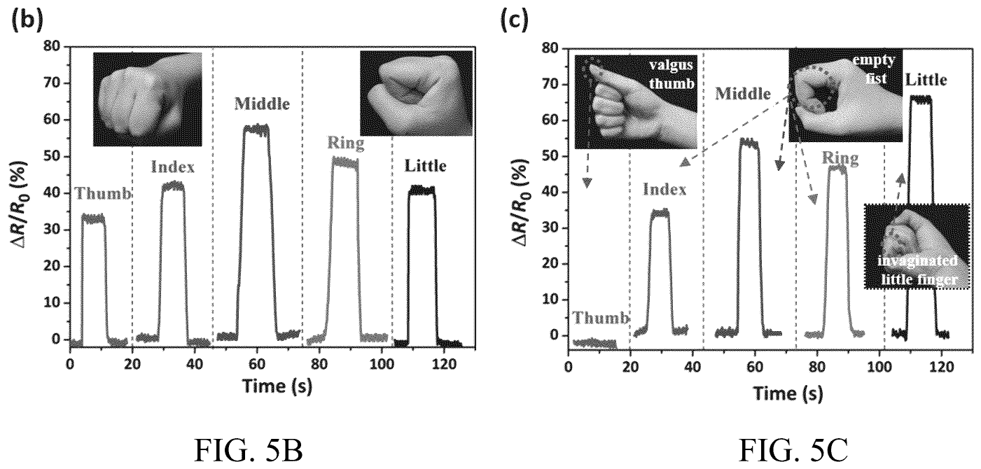

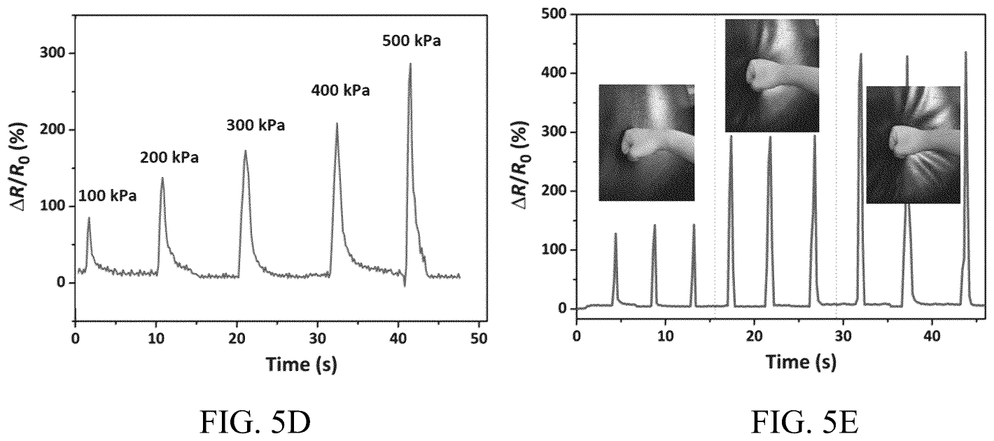

FIGS. 5A-5E illustrate a LM sensor for decoding the clenching posture and hitting strength of a punch in boxing training. FIG. 5A illustrate a photograph of an flexible tactile glove integrated with 3D-printed sensors. FIG. 5B illustrates relative resistance change of the sensors in response to a correct clenching posture. FIG. 5C illustrates relative resistance change of the sensors in response to an “incorrect” (or “wrong”) clenching posture. FIG. 5D illustrates electrical signals of the sensor at increased pressures. FIG. 5E illustrates relative resistance change of the sensor in response to various punching strength.

As a proof of concept, the sensing arrays are printed and assembled onto every finger of a flexible tactile glove 504 to decode fist clenching postures and impacting strengths (see FIG. 5A). The bending angles of all five fingers can be obtained from the measured piezoresistive signals (see FIG. 5B), which could be used to distinguish between correct and incorrect fist clenching postures in real time. Compared with the relative resistance changes of the professional fist clenching method, incorrect fist gestures such as the valgus thumb, empty fist and invaginated little finger can be identified and distinguished using the real-time electrical signals (see FIG. 5C). In addition to the clenching posture, the strength of punching is also crucial to training and competition. The increase in the punching force leads to the enhanced sensing response (see FIG. 5D), providing real-time visual feedback to trainees and coaches. FIG. 5E illustrates that the sensory glove demonstrates excellent reversibility and reliability during punch-release cycles, without noticeable degradation in sensing signals.

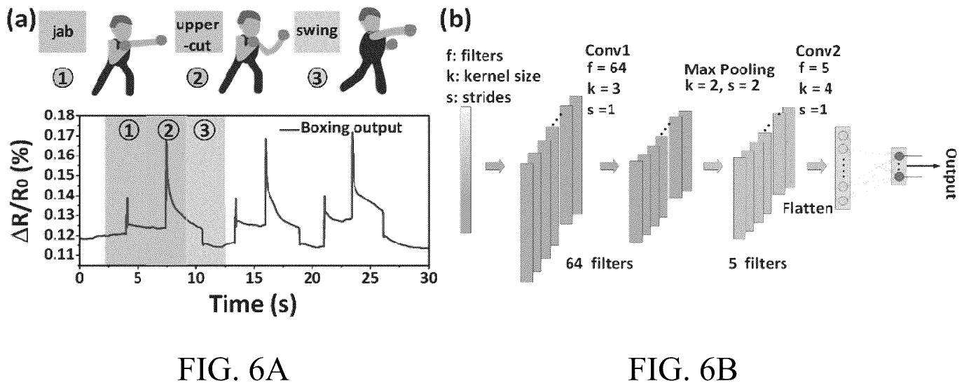

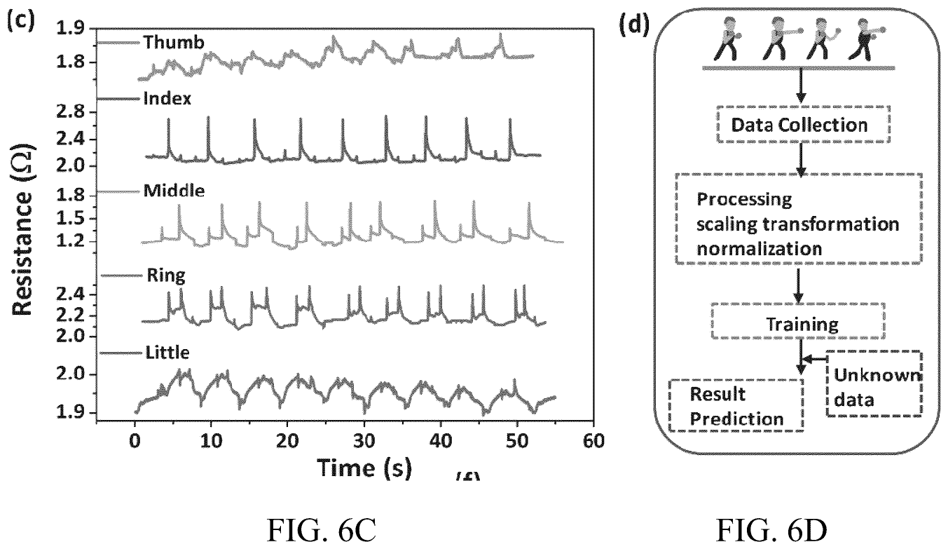

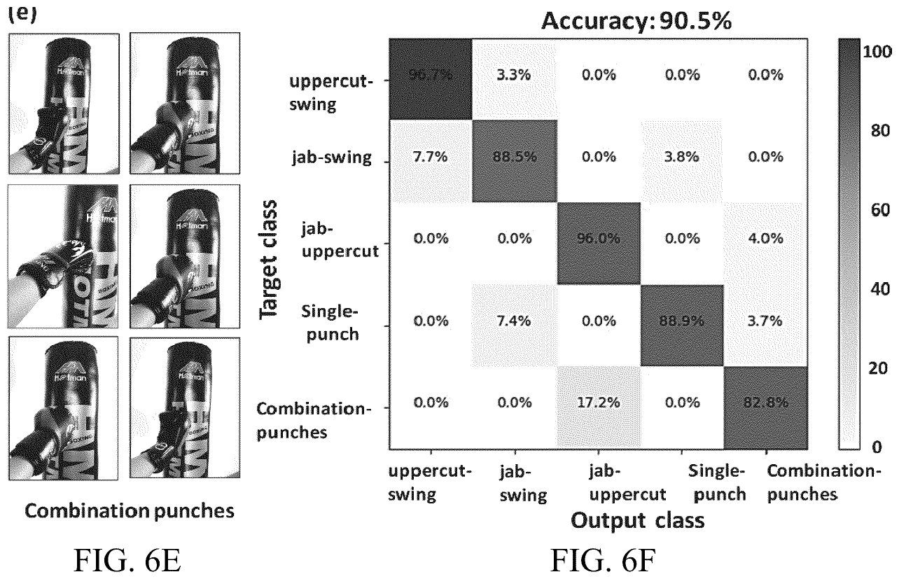

Since the tactile glove is capable of decoding clenching postures and punching strength, it paves the way for the development of an intelligent boxing recognition system, in which various types of punching techniques (e.g., jab, swing, uppercut, and combination punches, FIG. 6A) can be distinguished in real time.

FIGS. 6A-6F illustrate boxing identification of tactile glove assisted with deep learning. FIG. 6A illustrates a schematic of three boxing punches and corresponding sensing signals. FIG. 6B illustrates a CNN architecture constructed for identifying boxing postures from tactile information input. FIG. 6C illustrates output resistance of five fingers corresponding to the jab, uppercut, and swing punches. FIG. 6D illustrates a schematic of a process for training and real-time identification. FIG. 6E illustrates photographs of combination punches used in the dataset. FIG. 6F illustrates a classification test confusion matrix of boxing recognition derived from the tactile glove.

For a unique demonstration towards an application in sports training, the smart tactile glove is worn by a boxer to evaluate its recognition capability aided by the deep-learning algorithm. A convolutional neural network (CNN) architecture of the intelligent boxing system (see FIG. 6B) is proposed to achieve precise identification boxing punches. The whole data (containing 150 sets, 30 sets for each punch) are divided into a training set (90%) and a testing set (10%) to train the CNN model. Given the complexity of the sensing signals acquired from five different fingers in different punches (see FIG. 6C), only the real-time resistance change of the middle finger is used (see FIG. 6D) to simplify the analysis. The confusion map of the classified result indicates that the boxing recognition accuracy of the five punches can reach 90.5% (see FIGS. 6E-6F). Certain embodiments of the present disclosure are applicable in smart sport-training, human-machine interface, and humanoid robotics.

### Conclusions and Additional Details of Experimental Procedures

The present disclosure provides (and demonstrates through the described experiments) a printable LM sensor with high sensitivity and excellent mechanical properties, via effective rheological modification of LM and strain redistribution mechanics tuned by nonconductive particles (e.g., SiO₂ particles, TiO₂ particles, Al₂O₃ particles, etc.). In certain embodiments, the incorporation of SiO₂ micro-particles increases the elastic modulus, viscous modulus, yield stress, and viscosity of LM, which enables rapidly customizable and scalable fabrication of soft electronics based on modified LMs through 3D printing. The inclusion of SiO₂ particles in LM leads to enhanced sensitivity at large tensile strains (the GF is 5.72 for strain range between 0 and 100%, 11.36 for 100-200%, and 23.91 for 200-300%) due to strain redistribution mechanics within the conductive LM wires, and superior mechanical robustness to endure extreme pressure (i.e., 0.9 MPa). As a proof-of-concept demonstration, the multifunctional sensor is integrated onto a tactile glove to decode the clenching posture and hitting strength in boxing training. This tactile glove is shown to be able to identify different boxing punches (ab, swing, uppercut, and combination punches) with a recognition accuracy of 90.5%, assisted by a deep learning algorithm. The excellent combination of mechanical and electrical properties and demonstration in wearable systems promise great potential of the LM sensors in a wide variety of applications, such as smart sport-training, robotic operation, and human-machine interface.

### Material Preparation

In certain experiments described herein, the eutectic metal alloy was synthesized by mixing gallium (75%) and indium (25%) (eGaIn, Sigma-Aldrich). It maintains liquid state at room temperature (15.7° C. melting point). SiO₂ micro-particles (40 μm diameter, and 6 μm diameter, Sigma-Aldrich) composing 0, 1, 3.5, 6, and 20 wt % were mixed into the eGaIn. The mixtures were stirred in the air at room temperature with 500 rpm for 2 min and 2000 rpm (vigorously stirred) for 8 min. After stirring, the mixtures containing eGaIn and uniformly distributed SiO₂ particle were obtained.

### Rheological Property Test

In certain experiments described herein, the rheological characterization was performed using an Anton Paar Physica MCR 301 setup. The rheometer was equipped with a Peltier plate and parallel plate geometry of 25 mm diameter. All the tests were carried out at 25° C.
(1) Preshear for 120 s at 1 s-1 to eliminate loading history.
(2) A frequency sweep at 0.5% strain from 0.1 to 500 rad s-1 was used to determine
elastic (G′) and viscous modulus (G″) of the composites.
(3) An amplitude sweep at 1 rad s-1 from 0.05 to 1000% strain was used to determine the linear viscoelastic region of the composites as well as two versions of oscillation yield stress (σy).
(4) A flow sweep from 0.01 to 10 s-1 was used to determine the shear rate dependent viscosity (η) of the material.

### FEA Simulation

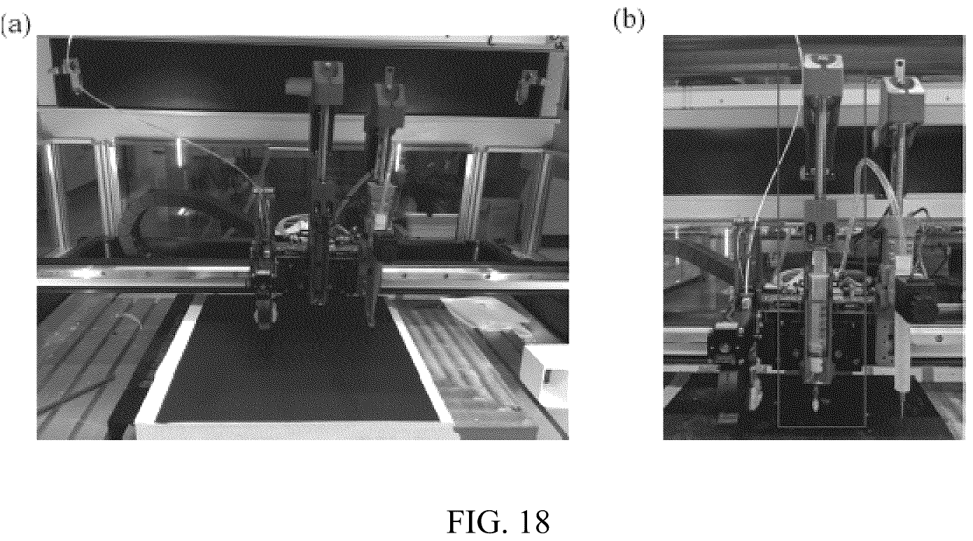

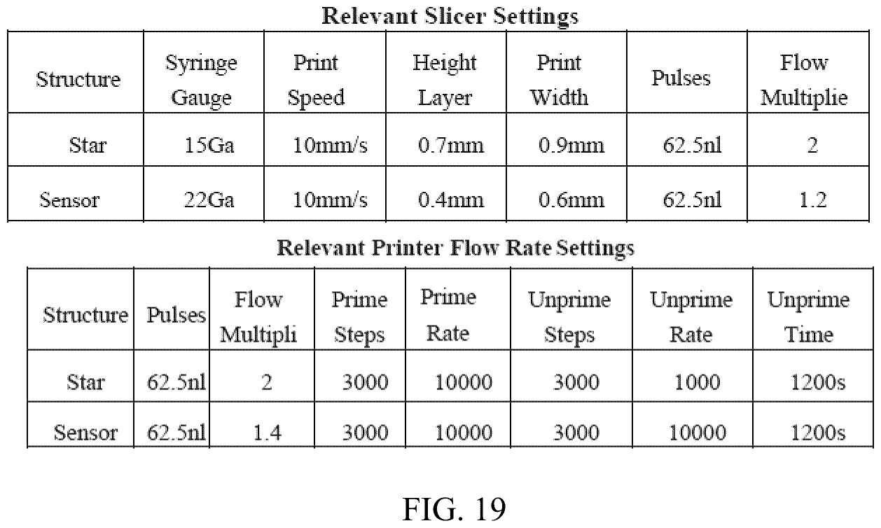

In certain experiments described herein, to investigate the mechanical deformation of the LM sensors, finite element analysis (FEA) was performed using ABAQUS. The Ecoflex and LM were modeled as Yeoh and Neo-Hookean hyperelastic materials using three-dimensional (3D) hybrid stress elements (C3D8H). The uniformly distributed SiO₂ particles were assembled into the LM wires by embedding constraints. SiO₂ particles were modeled using elastic isotropic material with 3D stress elements (C3D8R), with a Young's modulus of 76 GPa and a Poisson's ratio of 0.3. The coefficients C10, C20, and C30 were 0.0104 MPa, 0.0072 MPa, and 0.012 MPa for Ecoflex, by fitting experimental data from uniaxial tensile test (see FIG. 17). The coefficient of LM is set as C10=0.5 kPa based on the shear modulus of 1 kPa.

### 3D Structure Printing

In certain experiments described herein, the composites with 1 wt. % 6 μm SiO₂ particles were used to print complicated 3D structures utilizing a commercial modular 3D printer (Hydra 640 from Hyrel 3D, see FIG. 17). The syringe extrusion head on the printer was from a regular 10 ml syringe and the syringe plunger was precisely controlled by a linear motor. Nozzles with diameters 15 and 22 Ga were used to print star and sensor shapes, respectively. The one-layer star and four-layer star were printed by dispensing the composites layer by layer at a printing speed of 10 mm s⁻¹ through a 15 Ga nozzle. The height of each layer was 0.7 mm. Besides, we directly printed strain sensors on a tactile glove over the first knuckle regions of each finger. Each sensor was 0.6 mm wide and 20 mm long, which was printed using a 22 Ga nozzle and a printing speed of 10 mm/s.

### Fabrication and Characterization of the Sensors

In certain experiments described herein, the Ecoflex (a 1:1 mixture of Ecoflex part A and part B, Ecoflex Supersoft 0030, smooth-on, Inc.) substrate was coated on the glass by spin coating (1500 rpm for 30 s) and curing at 80° C. for 30 min. The LM-SiO₂ composite wires were fabricated by printing onto the substrate. After the copper wires were attached to the printed LM wires, another Ecoflex encapsulation layer was coated on the top and cured at 80° C. for 30 min. The sample was cut into strips (40 mm×20 mm×1 mm) and carefully peeled off of the glass to obtain the sensors. To characterize the electrical response, the sensing signals from the sensors were obtained using a semiconductor parameter analyzer (4200-SCS, Keithley).

### Mechanical Test

In certain experiments described herein, the tension and compression tests were carried out using an Instron mechanical testing system (e.g., using Instron LEGEND2345). The speed of the tensile test and tensile cyclic test were both 240 mm/min. Tensile cycling tests were performed 100 times under 100% strain. The speed of the compression test and compression cyclic test were both 2 mm/min. Compression cycling tests were carried out 100 times under 100 kPa pressure.

### ENUMERATED EMBODIMENTS

The following exemplary embodiments are provided, the numbering of which is not to be construed as designating levels of importance:

Embodiment 1: A liquid metal sensor comprising a wire, the wire including a liquid metal (LM) composite material, the LM composite material including: a LM material; and a nonconductive material.

Embodiment 2: The sensor of Embodiment 1, wherein the nonconductive material is SiO₂, wherein the LM composite material is LM-SiO₂.

Embodiment 3: The sensor of Embodiment 1, wherein the nonconductive material includes a particle size within the range of 10 nm-500 μm.

Embodiment 4: The sensor of Embodiment 1, wherein the LM material is a low melting temperature material.

Embodiment 5: The sensor of Embodiment 1, wherein the LM material is a gallium-indium alloy with a weight percent (wt %) Ga:In of about 75:25.

Embodiment 6: The sensor of Embodiment 1, wherein the LM composite material has an improved material property relative to the LM material, wherein the improved material property is selected from the group consisting of: modulus of elasticity; shear modulus; yield stress; oscillation yield stress; shear yield stress; and viscosity.

Embodiment 7: The sensor of Embodiment 1, wherein the nonconductive material is distributed uniformly throughout the LM composite material, wherein the nonconductive material is selected from the group consisting of: oxide particles; polymer coated conductive particles; polymer coated nonconductive particles; oxide coated conductive particles; oxide coated nonconductive particles; and polymer particles.

Embodiment 8: The sensor of Embodiment 7, wherein the oxide particles are selected from the group consisting of: SiO₂, TiO₂, Al₂O₃.

Embodiment 9: The sensor of Embodiment 1, wherein the LM material includes a material selected from the group consisting of: a gallium alloy; an indium alloy; a gallium-indium alloy; Eutectic gallium-indium (EGaIn); a Galinstan (GaInSn) alloy; a Field's alloy; and a Bismuth based Eutectic alloy.

Embodiment 10: The sensor of Embodiment 1, further comprising: an attachment mechanism configured to attach the wire to a human to monitor movement or pressure.

Embodiment 11: The sensor of Embodiment 10, wherein the attachment mechanism is a glove including a containment element configured to contain the wire.

Embodiment 12: The sensor of Embodiment 1, further comprising: a plurality of wires, wherein each of the plurality of wires includes the LM composite material.

Embodiment 13: The sensor of Embodiment 12, wherein each of the plurality of wires is configured to monitor movement of a different location.

Embodiment 14: The sensor of Embodiment 1, wherein the sensor is configured to communicate motion information to a computer system.

Embodiment 15: The sensor of Embodiment 14, wherein the computer is configured to implement a convolutional neural network (CNN) architecture to determine a motion profile.

Embodiment 16: The sensor of Embodiment 1, further comprising: a first terminal located at a first end of the wire; and a second terminal located at a second end of the wire.

Embodiment 17: A method of monitoring motion with any of the sensors of Embodiments 1-16, the method comprising:
(a) providing the wire to a portion of a human body to monitor motion;
(b) applying an electrical signal through the wire;
(c) monitoring the electrical signal; and
(d) determining a motion profile based on material and geometric properties of the wire.

Embodiment 18: The method of Embodiment 17, wherein the determining includes at least one of the group consisting of: processing, scaling transformation, and normalization.

Embodiment 19: The method of Embodiment 17, wherein the determining includes using a convolutional neural network (CNN) architecture to determine the motion profile.

Embodiment 20: A method of modifying rheological properties of liquid metal sensor, the method comprising:
(a) providing a liquid metal (LM) material; and
(b) integrating a nonconductive material into the LM material to form a LM composite material.

Embodiment 21: The method of Embodiment 20, wherein the nonconductive material is SiO₂, wherein the LM composite material is LM-SiO₂.

Embodiment 22: The method of Embodiment 20, wherein the nonconductive material includes a particle size within the range of 10 nm-500 μm.

Embodiment 23: The method of Embodiment 20, wherein the LM material is a low melting temperature material.

Embodiment 24: The method of Embodiment 20, wherein the LM material is a gallium-indium alloy with a weight percent (wt %) Ga:In of about 75:25.

Embodiment 25: The method of Embodiment 20, wherein the LM composite material has an improved material property relative to the LM material, wherein the improved material property is selected from the group consisting of: modulus of elasticity; shear modulus; yield stress; oscillation yield stress; shear yield stress; and viscosity.

Embodiment 26: The method of Embodiment 20, wherein the nonconductive material is distributed uniformly throughout the LM composite material, wherein the nonconductive material is selected from the group consisting of: oxide particles; polymer coated conductive particles; polymer coated nonconductive particles; oxide coated conductive particles; oxide coated nonconductive particles; and polymer particles.

Embodiment 27: A system for recognizing personal activities, comprising:
a liquid metal (LM) sensor configured to measure motion or pressure, the LM sensor including a wire made from a LM composite material, the LM composite material including a LM material and an nonconductive material;
a computer system in electronic communication with the LM sensor, the computer system being configured to determine a plurality of motion profiles from motion signals from the LM device, the computer system including a convolution neural network configured to be trained to differentiate each of the plurality of motion profiles.

Embodiment 28: The system of Embodiment 27, further comprising an attachment mechanism configured to attach the LM sensor to a body.

Embodiment 29: The system of Embodiment 27, wherein the attachment mechanism is a glove.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

What is claimed is:

1. A liquid metal sensor comprising a wire, wherein the wire includes a liquid metal (LM) composite material, wherein the LM composite material includes:
a LM material; and
a nonconductive material.

2. The sensor of claim 1, wherein the nonconductive material is SiO₂, and wherein the LM composite material is LM-SiO₂.

3. The sensor of claim 1, wherein the nonconductive material includes a particle size within the range of 10 nm-500 km.

4. The sensor of claim 1, wherein at least one of the following applies:
the LM material is a low melting temperature material;
the LM material is a gallium-indium alloy with a weight percent (wt %) Ga:In of about 75:25.

5. The sensor of claim 1, wherein the LM composite material has an improved material property relative to the LM material, wherein the improved material property is selected from the group consisting of: modulus of elasticity; shear modulus; yield stress; oscillation yield stress; shear yield stress; and viscosity.

6. The sensor of claim 1, wherein the nonconductive material is distributed uniformly throughout the LM composite material, wherein the nonconductive material is selected from the group consisting of: oxide particles; polymer coated conductive particles; polymer coated nonconductive particles; oxide coated conductive particles; oxide coated nonconductive particles; and polymer particles;
optionally wherein the oxide particles are selected from the group consisting of: SiO₂, TiO₂, and Al₂O₃.

7. The sensor of claim 1, wherein the LM material includes a material selected from the group consisting of: a gallium alloy; an indium alloy; a gallium-indium alloy; Eutectic gallium-indium (EGaIn); a Galinstan (GaInSn) alloy; a Field's alloy; and a Bismuth based Eutectic alloy.

8. The sensor of claim 1, further comprising an attachment mechanism configured to attach the wire to a human to monitor movement or pressure;
optionally wherein the attachment mechanism is a glove including a containment element configured to contain the wire.

9. The sensor of claim 1, further comprising a plurality of wires, wherein each of the plurality of wires includes the LM composite material;
optionally wherein each of the plurality of wires is configured to monitor movement of a different location.

10. The sensor of claim 1, wherein the sensor is configured to communicate motion information to a computer system;
optionally the computer is configured to implement a convolutional neural network (CNN) architecture to determine a motion profile.

11. The sensor of claim 1, further comprising:
a first terminal located at a first end of the wire; and
a second terminal located at a second end of the wire.

12. A method of monitoring motion with a sensor of claim 1, the method comprising:
(a) providing the wire to a portion of a human body to monitor motion;
(b) applying an electrical signal through the wire;
(c) monitoring the electrical signal; and
(d) determining a motion profile based on material and geometric properties of the wire.

13. The method of claim 12, wherein the determining includes at least one of the group consisting of: processing, scaling transformation, and normalization.

14. The method of claim 13, wherein the determining includes using a convolutional neural network (CNN) architecture to determine the motion profile.

15. A method of modifying rheological properties of liquid metal sensor, the method comprising:
(a) providing a liquid metal (LM) material; and
(b) integrating a nonconductive material into the LM material to form a LM composite material.

16. The method of claim 15, wherein at least one of the following applies:
the nonconductive material is SiO₂;
the LM composite material is LM-SiO₂;
the nonconductive material includes a particle size within the range of 10 nm-500 μm;
the LM material is a low melting temperature material;
the LM material is a gallium-indium alloy with a weight percent (wt %) Ga:In of about 75:25.

17. The method of claim 15, wherein the LM composite material has an improved material property relative to the LM material, wherein the improved material property is selected from the group consisting of: modulus of elasticity; shear modulus; yield stress; oscillation yield stress; shear yield stress; and viscosity.

18. The method of claim 15, wherein the nonconductive material is distributed uniformly throughout the LM composite material, wherein the nonconductive material is selected from the group consisting of: oxide particles; polymer coated conductive particles; polymer coated nonconductive particles; oxide coated conductive particles; oxide coated nonconductive particles; and polymer particles.

19. A system for recognizing personal activities, the system comprising:
a liquid metal (LM) sensor configured to measure motion or pressure, the LM sensor including a wire made from a LM composite material, the LM composite material including a LM material and an nonconductive material;
a computer system in electronic communication with the LM sensor, the computer system being configured to determine a plurality of motion profiles from motion signals from the LM device, the computer system including a convolution neural network configured to be trained to differentiate each of the plurality of motion profiles.

20. The system of claim 19, further comprising an attachment mechanism configured to attach the LM sensor to a body;
optionally wherein the attachment mechanism is a glove.
